(19) United States
(12) Patent Application Publication
(10) Pub. No.: US 2010/0222621 A1
(43) Pub. Date: Sep. 2, 2010

(54) Oxydehydrogenation of Ethylbenzene Using Mixed Metal Oxide or Sulfated Zirconia Catalysts to Produce Styrene

(76) Inventors: Anne May Gaffney, West Chester, PA (US); Ruozhi Song, Wilmington, DE (US); Chuen Yuan Yeh, Edison, NJ (US); Philip Jay Angevine, Woodbury, NJ (US)

Correspondence Address: Osha Liang LLP / Lummus, Two Houston Center, 909 Fannin, Suite 3500, Houston, TX, 77010, US

(21) Appl. No.: 12/394,353

(22) Filed: Feb. 27, 2009

(51) Int. Cl. C07C 5/48; C07C 5/42

(52) U.S. Cl. 585/441; 585/440; 585/444; 585/443

(57) ABSTRACT

Catalysts and methods are described for the dehydrogenation of ethylbenzene in the presence of an oxidant gas, such as oxygen or carbon dioxide, using a mixed metal oxide (MMO) catalyst or lithium-promoted sulfated zirconia catalyst to prepare styrene monomer. Ethylbenzene, steam or other inert gas, and an oxidant gas are fed to an oxydehydrogenation unit containing a MMO catalyst or lithium-promoted sulfated zirconia catalyst to produce a dehydrogenated product mixture. The dehydrogenated product mixture is cooled, off gases and condensate are separated from the mixture, and the dehydrogenated product mixture is fed to a distillation unit. Styrene monomer is distilled from the dehydrogenated product mixture.

### FIELD OF THE INVENTION

This invention relates to catalyst compositions and methods for oxydehydrogenation of ethylbenzene for the production of styrene. The catalysts used in the process may be either mixed metal oxides or sulfated zirconia.

### BACKGROUND

Styrene monomer is an important petrochemical used as a raw material for thermoplastic polymer products such as synthetic rubber, ABS resin and polystyrene. Over 90% of the styrene monomer produced today is made by dehydrogenation of ethylbenzene (EB). EB is prepared by the alkylation of benzene, available as a refinery product, with ethylene typically obtained from the cracking or dehydrogenation of ethane.

In the most common commercial process used today, styrene monomer is produced by dehydrogenation of ethylbenzene (EB) in the presence of excess steam over a potassium-promoted iron oxide catalyst. The dehydrogenation step is performed by adding excess superheated steam to EB in an adiabatic reactor under pressurized conditions with a reaction temperature of about 600° C. Although very selective to styrene, this technology has some inherent limitations, including thermodynamic limitations, low conversion rates, required recycling of unconverted reactants, highly endothermic heat of reaction and catalyst deactivation by coking. In this process, the ethylene stream accounts for about 40% of the raw material costs of EB, and superheated steam accounts for an estimated 10% of the cost for styrene production

The economics of this process can be improved by the ABB/UOP Smart™ process. In this process, the hydrogen formed during the dehydrogenation of the EB is oxidized with oxygen to water. The removal of hydrogen from the process shifts the reaction equilibrium in the dehydrogenation unit to substantially increase single-pass EB conversions while maintaining high styrene monomer selectivity. The heat generated by the oxidation of the hydrogen is used to reheat the reaction mixture, eliminating costly interstage heat exchange equipment and reducing superheated steam requirements. EB conversions of about 80% can be achieved with this method.

In a process described in U.S. Pat. No. 6,958,427, ethylbenzene is dehydrogenated to styrene in the presence of carbon dioxide as a soft oxidant over a catalyst comprising vanadium and iron. Compared with the conventional process, the presence of carbon dioxide allows operation at a lower temperature and provides enhanced conversion and significant energy savings. However, severe catalyst deactivation by carbon dioxide has limited commercialization of this process.

The use of oxygen in ethylbenzene ODH has been the object of considerable research over the last five decades. By co-feeding oxygen with ethylbenzene, water is produced rather than hydrogen, resulting in a reaction that is markedly exothermic and thermodynamically favorable. The advantage of this route is that the reaction can be operated at lower temperatures, without steam co-feed, and some secondary cracking can be avoided that leads to toluene, benzene and coke. However, low styrene selectivity has limited with low EB conversions, leading to total oxygen consumption, and has limited its commercialization; clearly, a more selective catalyst needs to be developed. In “Appl. Catal. A, 1995, pp. 219 to 239”, Cavani et al reviewed the catalysts and their performance for the oxidative dehydrogenation of ethylbenzene to styrene in the presence of oxygen.

The use of certain mixed metal oxide catalysts and Li-doped sulfated zirconia catalysts for oxydehydrogenation of ethane to ethylene has been reported in U.S. Patent Publication No. 2005/0085678 and Suzuki et al., Chem. Commun. 1999, p. 103 to 104. In U.S. Pat. No. 5,336,822, respectively. A mixed metal oxide catalyst having antimony and tin for use in oxydehydrogen of 4-vinyl-1-cyclohexene to styrene using oxygen has also been reported. None of these references describes the use of a mixed metal oxide catalysts and Li-doped sulfated zirconia catalysts for oxydehydrogenation of ethylbenzene to styrene.

Due to the potential advantages over the prior art, ethylbenzene ODH to styrene has been the object of considerable research. Over the years, many catalyst systems have been investigated including carbon molecular sieves, metal phosphates, and other catalysts. However, commercialization been limited due to low ethylbenzene conversion, low product selectivity, and often rapid, catalyst deactivation. As such, there exists an ongoing and unmet need in the industry for less expensive and more efficient methods for styrene production.

### SUMMARY OF THE INVENTION

The present invention relates to an improved process for the production of styrene monomer by the oxidative dehydrogenation (“oxydehydrogenation”) of ethylbenzene in the presence of a mixed metal oxide (MMO) catalyst, or a sulfated zirconia catalyst. Generally, MMO catalysts are used in processes using oxygen as an oxidizing gas, and a sulfated zirconia catalyst is used when the oxidizing gas is carbon dioxide or a combination of carbon dioxide and oxygen.

In one aspect the invention relates to a catalyst composition for use in the dehydrogenation of EB in the presence of an oxidizing agent or oxidant (i.e., oxidative dehydrogenation). The catalyst is preferably one of: (1) a MMO comprising molybdenum, vanadium, tellurium, niobium and a promoter, (2) a MMO comprising antimony and tin with one or more promoters, or (3) a sulfated zirconia with a lithium promoter.

In another aspect the present invention relates generally to a process for producing styrene by dehydrogenation of ethylbenzene in an oxydehydrogenation (ODH) unit. In one aspect, ethylbenzene is combined with an oxidant and a gas such as CO₂, N₂ or superheated steam in the presence of a MMO catalyst in an ODH unit to produce styrene monomer. In this case, the CO2 is inert due to the low process temperatures (about 400° C.).

In another aspect, a the sulfated zirconia catalyst in an ODH unit to produce styrene monomer. In this case, the feed to the ODH unit will consist of: (1) ethylbenzene and CO₂; (2) ethylebenzene, CO₂ and steam; (3) ethylebenzene, CO₂ and O₂; or (4) ethylbenzene, CO₂, O₂ and steam. In this case, the CO2 acts as an oxidant and a diluent, and the small amount of steam needed is for selectivity improvement only.

Intermediate oxidants, such as NO₂, may also be used with either catalyst.

A mixture of dehydrogenated products is produced in the ODH unit. The dehydrogenated mixture is cooled in one or more heat exchangers. Preferably, heat is recovered during the cooling process for use in the process. The cooled dehydrogenated mixture is sent to a separation unit followed by a distillation unit to separate and recover the styrene monomer products and by-products. The product stream containing styrene monomer is removed and sent for further processing or packaging.

The catalyst used in the dehydrogenation unit may be a mixed metal oxide catalyst or a sulfated zirconia catalyst. When a mixed metal oxide catalyst is used, the oxidizing agent may be oxygen, which may be provided as air. When the catalyst is a sulfated zirconia catalyst, the oxidizing agent may be carbon dioxide or a mixture of carbon dioxide and oxygen.

One advantage of the present invention is that the ODH reaction requires less energy input than other dehydrogenation processes. Because the operating conditions in the ODH reactor are lesse severe and may be performed in the presence of oxygen, coking of the catalyst is reduced. Furthermore, the process results in higher EB conversion, thereby enabling higher throughput and superior catalyst performance, resulting in higher product yield, less recycle EB and longer catalyst life. These advantages are given by way of non-limiting example only, and additional benefits and advantages will be readily apparent to those skilled in the art in view of the description set forth herein.

### BRIEF DESCRIPTION OF THE DRAWINGS

FIG. 1 is a schematic flow chart illustrating an embodiment of the process of the present invention for producing styrene monomer by oxydehydrogenation of ethylbenzene.

### DETAILED DESCRIPTION OF THE INVENTION

The present invention relates to a process for the dehydrogenation of ethylbenzene (EB) in the presence of an oxidant, for example, oxygen (O₂), carbon dioxide (CO₂) or combinations thereof. The process is referred to as an “oxydehydrogenation” process, or ODH. The process takes place in the presence of a catalyst such as one of the catalysts described in detail below.

In one embodiment, the catalyst used in the process is a mixed metal oxide (MMO). In a preferred embodiment, the MMO catalyst comprises molybdenum (Mo), vanadium (V), tellurium (Te), and niobium (Nb) and one or more promoters, A, selected from the group of Cu, Ta, Sn, Se, W, Ti, Fe, Co, Ni, Cr, Zr, Sb, Bi, Pd, Pt, an alkali metal, an alkaline-earth metal and a rare earth. As used herein, “promoter” means an accelerator of catalysis, but not a catalyst by itself. At least one element selected from Mo, V, Te, and Nb is present in the form of an oxide. In this embodiment of the process, the catalyst comprises the formula: MoVₐTe_{b}Nb_{c}A_{d}Oₓ, where a, b, c, d, and x represent the gram atom ratios of the elements relative to Mo. In preferred embodiments, a, b and c have values lying between about 0.001 and about 4.0, d is between about 0.0001 and about 2.0 and x depends upon the valence of the elements Mo, V, Te and Nb. The composition, structure and method of preparing this catalyst is described in U.S. Patent Publication No. 2005/0085678, the contents of which are hereby incorporated in their entirety.

In another embodiment of the process, the MMO catalyst comprises antimony (Sb), tin (Sn) and oxygen in combination with one or more of the promoters described above. The molar ratio of tin to antimony is generally in the range from about 1:1 to about 20:1. The promoter is present in an amount of between 0.001 and 1.0 relative to the amount of tin in the catalyst. The composition, structure and method of preparing this catalyst is described in detail in U.S. Pat. No. 5,366,822, the contents of which are hereby incorporated in their entirety.

In yet another embodiment, the catalyst comprises sulfated zirconia (Zr) with a lithium promoter. The composition, structure and method of preparing this catalyst are described in detail in Suzuki et al., “Chem. Commun.” 1999, pages 103 to 104, the contents of which are hereby incorporated in their entirety.

Any of the MMO catalyst compositions may be provided on a solid support, for example, silica, alumina, a carbide, titanium oxide, cermet, ceramic, or mixtures thereof. The invention is not limited in this regard, and any appropriate solid support material may be used. In one embodiment, the solid support is present at from about 10% by weight to about 80% by weight with respect to the total weight of the catalyst. In a preferred embodiment in which oxygen is used as the oxidant in the process, a MoVTeNb MMO catalyst of the formula described above is provided on a solid support.

The catalysts used in the process of the present invention can be prepared by conventional methods. For example, the catalysts may be prepared starting from solutions of compounds of the different catalyst components, from solutions of the pure components themselves, or mixtures of both, with the desired atomic ratios. Typically, aqueous solutions of the catalyst components are prepared. Solutions containing the various components of the catalyst may be mixed, the solutions dried to a solid, and the resulting solid may be calcined to produce the desired catalyst. The mixing stage can be done starting from the compounds of the different elements, starting from the actual pure elements in solution, or by hydrothermal methods. The drying stage can be carried out by conventional methods, for example, in a kiln, evaporation with stirring, evaporation in a rotavapor, vacuum drying, or spray drying.

Following drying, the catalyst material may be calcined by conventional methods. For example, the calcination stage of the dry solid can be carried out in an inert gas atmosphere, such as nitrogen, helium, argon or mixtures of these gases, or may be carried out in air or mixtures of air with other gases. The MMO catalyst may require two stage calcinations, first in air, then in N₂ or other inert environment. The calcination stage can be carried out (a) by flowing inert gas over the catalyst material (with spatial velocities between 1 and 400 h⁻¹) or (b) statically.

In any of the embodiments disclosed herein, the promoter may be of any type generally recognized by those of ordinary skill in the art including, for example, lithium (Li), phosphorus (P), zinc (Zn), copper (Cu), lead (Pb), germanium (Ge), selenium (Se), indium (In), tin (Sn), Ta, Sn, Se, W, Ti, Fe, Co, Ni, Cr, Zr, Sb, Bi, Pd, Pt, an alkali metal, an alkaline-earth metal and a rare earth. The promoter may be added to the catalyst components at the mixing stage and incorporated into the catalyst composition. Alternatively, the promoter may be added to the catalyst material between calcination steps.

As described above, the MMO catalyst can be supported on a solid such as: silica, alumina, titanium oxide, carbide or mixtures thereof, for example, silicon carbide. In these cases, the fixing of the different elements of the catalyst on the support can be achieved by conventional methods, e.g. incipient wetness, impregnation, excess solution impregnation/ion exchange, or simply by precipitation.

In the process of the present invention, dehydrogenation of ethylbenzene is performed to produce styrene monomer using the catalyst compositions described above in the presence of an oxidizing agent such as oxygen, carbon dioxide or mixtures thereof. The process of the present invention generally comprises the steps of: a) feeding to an ODH unit containing one of the catalysts described above EB, steam or other inert gas as a diluent, and an oxidant, such as for example oxygen, carbon dioxide or combinations thereof to produce a dehydrogenation product mixture; b) cooling the dehydrogenation mixture and feeding the cooled mixture to a separator; c) separating the dehydrogenation mixture from gases and condensed steam; d) sending the dehydrogenation mixture to a distillation unit, where the excess EB is removed and recycled to the ODH unit, and the styrene monomer is separated from by-products, such as, for example, toluene and benzene.

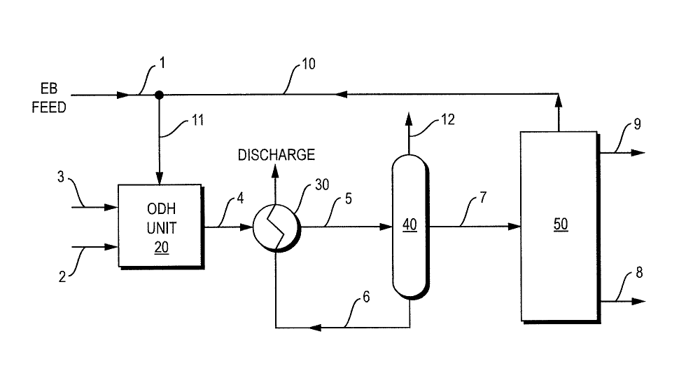

In one embodiment of the process, shown schematically in FIG. 1, the process may be performed as follows. Fresh EB feed (1) is combined with recycle EB (10) from a distillation unit (50) to form EB feed stream (11). The EB feed stream (11) is fed to an ODH unit (20) with steam or other diluent gas feed (3) and oxidant gas feed (2). The ODH unit (20) contains one of the catalysts for oxydehydrogenation of EB as described above, either MMO or sulfated zirconia. In one embodiment, the ODH unit contains an MMO catalyst and oxygen is used as the oxidizing gas. In another embodiment, sulfated zirconia is used as the catalyst and the oxidizing gas is carbon dioxide or a mixture of carbon dioxide and oxygen.

The dehydrogenation reaction is preferably carried out in the gaseous phase in a fixed-bed, a moving-bed or a fluid-bed catalytic reactor. Fluid-bed reactors are preferred for their technological advantages which are well known to those skilled in the field. The reaction temperature is preferably between about 200° C. and 650° C. The ODH unit is preferably operated at a pressure of between about 1 bar and about 15 bar. In one preferred embodiment, the reaction temperature is between about 300° C. and 450° C. using oxygen as the oxidizing gas and an MMO catalyst. In another preferred embodiment, the reaction temperature is between about 500° C. and 650° C. using carbon dioxide as the oxidizing gas and a sulfated zirconia catalyst. The contact time, defined as the ratio between the volume of catalyst and the total flow of supply gases, is preferably between about 0.001 and 100 seconds. Although the contact time depends on the preparation method and composition of the catalyst used, in general the contact time is between 0.05 and 50 seconds. In some embodiments, the contact time is between 0.1 and 25 seconds. In yet other embodiments, the contact time is between 0.5 to 10 seconds.

Styrene momomer is produced in the ODH unit (20) and exits the ODH unit as a dehydrogenation product stream (4). In addition to styrene monomer, the dehydrogenation product stream typically contains excess EB and reaction by-products. The process differs from conventional ethylbenzene dehydrogenation in that CO, CO₂ and H₂O will be the major by-products, and organics such as benzaldehyde, benzoic acid or acetophenone will be present as minor by-products. Benzene and toluene may not be formed at all in the process. The dehydrogenation product stream (4) is cooled in a heat exchanger (30) by heat exchange contact with a cooling stream (6) to produce a cooled dehydrogenation product stream (5). In the embodiment shown in FIG. 1, a condensate stream from separator (40) is used as the cooling stream, however, any source of cooling water may be used in the heat exchanger. In a preferred embodiment, the cooling water is heated in heat exchanger (30) and generates steam for use in the ODH unit. More than one heat exchanger may be used in the process to cool the dehydrogenation product stream (4).

The cooled dehydrogenation product stream (5) is sent to a separator (40) where off gases (12) and condensate (6) are separated from the dehydrogenation product stream. The off gases may be sent to a boiler and burned to generate steam for the process. The dehydrogenation products are sent through line (7) to a distillation unit (50) to separate the styrene monomer product (8) from excess EB (10) and by-products (9). Excess EB is removed and recycled to the ODH unit (20) through recycle line (10). The by-products typically will include CO, CO₂ and H₂O will be the major by-products, and organics such as benzaldehyde, benzoic acid or acetophenone will be present as minor by-products. If desired, these by-products can themselves be separated into a plurality of by-product lines and fed to other processes.

The oxidant gas fed to the ODH unit may be provided as air, oxygen, carbon dioxide, or a mixture thereof. Oxygen and carbon dioxide represent the boundaries of highly reactive and mildly reactant oxidants, respectively. If desired, and intermediate oxidant, such as for example NO₂ may be used as well. The oxidant gas may be fed into the oxydehydrogenation (ODH) unit as a single stream or at several injection points along the catalyst bed. Recycled ethylbenzene may also be added at this point. In embodiments of the process in which a fixed bed reactor is used and oxygen is the oxidizing agent, the oxygen concentration will typically be maintained in the range of 8-10 mole % to avoid forming a flammable or explosive mixture. In embodiments using a fluidized bed reactor, higher concentrations of oxygen, up to 40 mole % or more, may be used. In embodiments in which CO₂ is used as the oxidizing agent, the CO₂ concentration may be be as high as needed. In one embodiment, the feed is a mixture of ethylbenzene and CO₂ with a 1:1 molar ratio.

The oxidizing agents may be introduced in the form of a gas containing molecular oxygen or carbon dioxide or both, which may be air or a gas richer or poorer in molecular oxygen and/or carbon dioxide than air, for example pure oxygen or pure carbon dioxide. A suitable gas may be, for example, oxygen or carbon dioxide or both diluted with a suitable diluent, for example nitrogen or helium.

One skilled in the art will recognize that numerous variations or changes may be made to the process described above without departing from the scope of the present invention. Accordingly, the foregoing description of preferred embodiments is intended to describe the invention in an exemplary, rather than a limiting, sense.

What is claimed is:

1. A process for the production of styrene, comprising the steps of:
a) feeding to an oxydehydrogenation unit containing one of a mixed metal oxide or a sulfated zirconia catalyst which is capable of catalyzing the oxidative dehydrogenation of ethylbenzene to form styrene a stream of ethylbenzene, a stream of an inert gas, and a stream containing an oxidant gas to produce a dehydrogenation product mixture;
b) cooling the dehydrogenation product mixture and feeding the cooled dehydrogenation product mixture to a separator;
c) separating the dehydrogenation product mixture from gases and condensed steam; and
d) feeding the dehydrogenation product mixture to a distillation unit and distilling the styrene monomer from the dehydrogenation product mixture.

2. The process of claim 1, wherein the inert gas is steam.

3. The process of claim 1, wherein the catalyst is a mixed metal oxide catalyst.

4. The process of claim 1, wherein the catalyst is a sulfated zirconia catalyst.

5. The process of claim 3, wherein the catalyst is a mixed metal oxide having the empirical formula MoVₐTe_{b}Nb_{c}A_{d}Oₓ, wherein A is selected from the group consisting of Cu, Ta, Sn, Se, W, Ti, Fe, Co, Ni, Cr, Zr, Sb, Bi, an alkali metal, an alkaline-earth metal and a rare earth, and wherein a, b and c may be the same or different and have values lying between 0.001 and 4.0, d is between 0.0001 and 2.0 and x depends upon the valence of the elements Mo, V, Te and Nb.

6. The process of claim 3, wherein the catalyst is a mixed metal oxide comprising antimony, tin, oxygen and at least one promoter, wherein the molar ratio of tin to antimony is in the range from about 1:1 to about 20:1.

7. The process of claim 6, wherein the promoter is present in an amount of between 0.001 to 1.0 relative to the amount of tin in the catalyst and the promoter is selected from the group consisting of Li, P, Zn, Cu, Pb, Ge, Se, In, Ta, Se, W, Ti, Fe, Co, Ni, Cr, Zr, Sb, Bi, an alkali metal, an alkaline-earth metal and a rare earth.

8. The process of claim 4, wherein the catalyst is a Li-doped sulfated zirconia.

9. The process of claim 3, wherein the oxidizing agent comprises oxygen.

10. The process of claim 8, wherein the oxidizing agent comprises carbon dioxide or a mixture of carbon dioxide and air.

11. The process according to claim 1, wherein in the oxydehydrogenation unit the molar ratio of ethylbenzene to oxidant gas is between 8 to 10 mole %.

12. The process according to claim 1, wherein the oxydehydrogenation unit is operated at a temperature of between about 200 and 650° C. and a pressure of between about 1 and about 15 bar.

13. A process for the production of styrene, comprising the steps of:
a) feeding to an oxydehydrogenation unit containing one of a mixed metal oxide or a sulfated zirconia catalyst a stream containing ethylebenzene, a stream containing steam, and a stream containing an oxidant gas to produce a dehydrogenation product mixture stream;
b) feeding the dehydrogenation product mixture stream from the oxydehydrogenation unit to a heat exchanger to cool the dehydrogenation product mixture;
c) feeding the cooled dehydrogenation product mixture stream to a separator;
d) separating off gas and condensate from the dehydrogenation product mixture;
e) feeding the dehydrogenation product mixture from the separator to a distillation unit;
f) distilling the dehydrogenation product mixture to produce a styrene monomer product stream.

14. The process of claim 13, further comprising the step of distilling the unreacted ethylbenzene from the dehydrogenation product mixture and recycling the unreacted ethylbenzene to the ODH unit.

15. The process of claim 14, further comprising the step of feeding the condensate removed from the dehydrogenation product mixture in the separator to the heat exchanger to cool the dehydrogenation product mixture stream from the oxydehydrogenation unit.

16. The process of claim 13, wherein the catalyst is a mixed metal oxide catalyst.

17. The process of claim 13, wherein the catalyst is a sulfated zirconia catalyst.

18. The process of claim 16, wherein the catalyst is a mixed metal oxide having the empirical formula MoVₐTe_{b}Nb_{c}A_{d}Oₓ, wherein A is selected from the group consisting of Cu, Ta, Sn, Se, W, Ti, Fe, Co, Ni, Cr, Zr, Sb, Bi, an alkali metal, an alkaline-earth metal and a rare earth, and wherein a, b and c may be the same or different and have values lying between 0.001 and 4.0, d is between 0.0001 and 2.0 and x depends upon the valence of the elements Mo, V, Te and Nb.

19. The process of claim 16, wherein the catalyst is a mixed metal oxide comprising antimony, tin, oxygen and at least one promoter, wherein the molar ratio of tin to antimony is in the range from about 1:1 to about 20:1.

20. The process of claim 19, wherein the promoter is present in an amount of between 0.001 to 1.0 relative to the amount of tin in the catalyst and the promoter is selected from the group consisting of Li, P, Zn, Cu, Pb, Ge, Se, In, Ta, Se, W, Ti, Fe, Co, Ni, Cr, Zr, Sb, Bi, an alkali metal, an alkaline-earth metal and a rare earth.

21. The process of claim 17, wherein the catalyst is a Li-doped sulfated zirconia.

22. The process of claim 16, wherein the oxidizing agent comprises oxygen.

23. The process of claim 21, wherein the oxidizing agent comprises carbon dioxide or a mixture of carbon dioxide and air.

24. The process according to claim 12, wherein the oxydehydrogenation unit is operated at a temperature of between about 200 and about 650° C. and at a pressure of between about 1 bar and about 15 bar.
